(19) United States
(12) Patent Application Publication
(10) Pub. No.: US 2019/0274890 A1
(43) Pub. Date: Sep. 12, 2019

(54) VACUUM DRESSING FOR THE ENHANCEMENT OF COMPRESSION AND DRAINAGE IN VACUUM THERAPY

(71) Applicant: JUAN MARQUEZ CANADA, GANDIA (VALENCIA) (ES)

(72) Inventor: JUAN MARQUEZ CANADA, GANDIA (VALENCIA) (ES)

(21) Appl. No.: 16/296,191

(22) Filed: Mar. 7, 2019

(30) Foreign Application Priority Data
Mar. 8, 2018 (ES) U201830314

(51) Int. Cl. A61F 13/00; A61M 27/00; A61M 1/00

(52) U.S. Cl. CPC A61F 13/00068; A61F 13/0216; A61M 1/0023; A61M 27/00

(57) ABSTRACT

Vaccum dressing for the enhancement of compression and drainage in vacuum therapy, of those, which are sealed by an adhesive sheet on which is coupled a suction tube connected to a vacuum pump, and which comprise below of said adhesive sheet an external component, which is a water-permeable open pore foam, and an internal component, which is attached to said external component, and which comprises at least one outer layer, which is attached to said external component, water vapor permeable and impermeable to water and an internal layer adherent to the skin and intended to contact the body surface, characterized in that said external component is constituted by a plurality of foam fragments adjacent to each other and distributed over the surface of the internal component and in the spaces between said foam fragments a plurality of areas devoid of external component is generated in said internal component.

### OBJECT OF THE INVENTION

The object of the present invention is the enhancement of compression and drainage in vacuum therapy by the application in wounds or injuries of a novel vacuum dressing developed from the “improved vacuum dressing applicable as a postoperative compression treatment” described and claimed in the international patent application POT/ES2010/000221, hereinafter IVD, and of which the author of the present invention is the inventor.

The present invention proposes a new vacuum dressing that has a retraction potential, after the application of vacuum, greater than that of the IVD, which implies an enhancement of compression and drainage under the>action of the new vacuum dressing.

### BACKGROUND OF THE INVENTION

The application of Vacuum Therapy on the bed of a wound or injury has been shown experimentally and clinically to reduce edema. However, it should be noted, also, that in any wound or injury not only there is edema at the level of said wound or injury, but also perilesional and even at a distance. However, until the publication of the IVD, none of the preexisting or conventional vacuum dressings, hereinafter CVDs, has allowed the satisfactory treatment of said edema distant from said wound or injury. Since these CVDs have been designed and conceived under the mistaken concept that Vacuum Therapy is exclusively a Negative Pressure Therapy, and, consequently, said CVDs have based their principles and mechanisms of action in the promotion of the external drainage of the liquid responsible of said lesional edema. So much so, that Vacuum Therapy, for more than 50 years, is better known as Negative Pressure Wound Therapy.

However, while it is true that the application of vacuum therapy on the body surface generates negative pressures at the level of the pores of vacuum dressings, it is not less important than at the contact points of the solid components, ie, the walls of the pores, of said vacuum dressings with the body surface, vacuum therapy generates positive pressures. This means that vacuum therapy could also be used not only for said external drainage promotion, but also to extend the indications for the application of vacuum therapy even on the cutaneous surface distant from said wound or injury, with the objective of promoting internal drainage, that is, lymphatic and venous drainage, even of said distant edema.

But this is faced with the fact that, unfortunately, the most frequent cutaneous complication of said CVDs, especially if their application was maintained for a prolonged period of time greater than 1 week, is the irritation, maceration or even formation of blisters in the skin that, accidentally, could remain in contact with the pores of said CVDs, pores through which the vacuum is directly transferred to the body surface.

Based on this, the IVD was designed to improve the Compression Therapy by Vacuum, with the aim of promoting internal drainage, even of edema distant from the injury, extending the application of vacuum therapy even on the cutaneous surface distant to said wound or injury, but, at the same time, avoiding such adverse skin effects. Thus, said IVD, antecedent of the present invention, is a vacuum dressing that has in common with said CVDs being sealed by an adhesive sheet on which a suction tube connected to a vacuum pump is coupled. However, unlike said CVDs, said IVD comprises, under the aforementioned sealing adhesive sheet, an external component that is an open pore foam, permeable to water, which is attached to a semipermeable internal component, where the outermost layer of said internal component, that is, the one that is attached to the external component, is permeable to water vapor and impermeable to liquids, and where the innermost layer of said internal component, that is, the one intended to contact the surface body, is adherent to the skin.

Thus, the IVD was designed for the reduction of edema, not only at lesional level but also perilesional and distant, promoting for this purpose the drainage of said edema, mainly, through exercising the compression on the body surface safely, without said adverse effects, by protecting it from the direct action of vacuum. To this end, the IVD interposes a semipermeable internal component between the pores of the external component and the body surface. Thus, the reduction of tissue edema with the IVD is carried out in the following manner:
In closed wounds, through:
   Internal drainage, via lymphatic-venous; thanks to said compression exercised by said IVD. As shown in FIG. 1 of PCT/ES 2010/000221, said compression exerted under the action of the vacuum by said IVD has two vectors: a positive sagittal one, represented by the vertical arrows, which is exerted by the walls of the pores of said external component; and another negative tangential, represented by the horizontal arrows, which is produced by the collapse of said pores under the action of the vacuum and the subsequent traction/drag on the cutaneous surface of said external component joined to the internal component, since said internal component is, in addition to semipermeable, adherent to the skin.
   Transpiration through the body surface; since said internal component is permeable to water vapor, since it is semipermeable, it will allow said body surface to “breathe”, avoiding the maceration of both the wound and the skin. On the other hand, since the internal component is impermeable to liquids, due to being semipermeable, it will effectively and safely protect the cutaneous surface from contact with the pores of the external component in order to avoid the adverse effects mentioned above. Thus, this double quality, permeable to water vapor and impermeable to liquids, means that the IVD can be maintained “in situ” for a very long period, even longer than a month.
   Possibility of incorporating the accessory tubular drain device shown in FIG. 2 of PCT/ES 2010/000221 of said IVD.
In open wounds, n addition to said lympho-venous internal drainage, of said transpiration or of said possibility of incorporating said tubular drainage device, through:
   Selective aspiration of the wound exudate simply by performing one or more perforations, for example, “in situ” with a needle or with a scalpel, through said internal component, exclusively at the level thereof coinciding with the bed of the wound. Said selective aspiration on the wound bed will prevent said cutaneous irritant adverse effects, thanks to the fact that the rest of the internal component will maintain said protective semipermeability. But, in addition, the protective action of the internal component will also extend to the wound bed; thus, it is important to point out that, although the internal component is adherent to the skin, it is not adherent in a moist environment; that is, it adheres to the skin, but not to the wound bed and, furthermore, said internal component will also act as a tissue protection factor, not allowing the newly formed granulation tissue to contact the pores of the external component. In this sense, the IVD allows that even the external drainage of secretions/lesion exudate is effected in a more effective way than with the CVDs, since the barrier-protection effect of the internal component prevents the clogging of said pores by the granulation tissue neoformed which, in turn, will facilitate drainage through said pores. Thus, even in the case of tunneled deep wounds, the sagittal and centripetal tangential compressions exerted by said IVD, both on the body surface overlying said tunnels and at a distance therefrom, will literally contribute to “squeezing” the tissue underlying said IVD. This will lead to the subsequent reduction and progressive collapse of said tunnels, with the expression of the exudate contained in said tunnels, and the drainage of said exudate both internally, lympho-venously, and externally, by selective aspiration through said perforations, as well as by evaporation through the semipermeable internal component.

It should be mentioned that the IVD can also incorporate, both in open and closed wounds, between the semipermeable outer layer and the internal adherent layer of said Internal Component, a central hydrophilic middle layer with high absorption capacity. The role of said layer is more relevant in the cases of application of vacuum therapy in the intermittent mode, as well as it can constitute a safety factor in the event of a vacuum failure, for example, by accidental removal of the aspiration tube, exhaustion of the battery or failure of the machine for another reason, since it can avoid that said loss of vacuum becomes an emergency, allowing to defer the change of said IVD.

In said IVD, the vacuum pressure is transmitted by said suction tube connected to said suction pump and said pressure is distributed, under said sealing sheet, directly inside the pores of the external component, causing the contraction thereof under the vacuum action; and, being attached to the internal component, the external component will transmit on said internal component a tangential and sagittal compression action; in this way, said internal component is going to be compressed “tracked/dragged” by the external component. And the internal component, being attached to the cutaneous surface, will transmit on said cutaneous surface a tangential and sagittal compression action; although, being semipermeable said internal component, simultaneously it will act as a protective factor of the cutaneous surface, protecting it from the direct action of the vacuum and, simultaneously, allowing said surface to transpire; which will allow the IVD to be in uninterrupted contact with this surface, not only at the lesional level, but also on the perilesional skin and even distant from said wound or injury for even weeks, thanks to the protective semipermeability exerted by the internal component.

Thus, the IVD has allowed to increase the indications of said vacuum therapy, not only to promote healing in losses of substance or in closed wounds, but also as a tool in those situations or injuries that were previously not susceptible to be treated with said vacuum therapy: as its anti-edema use after liposuction, as a selective compression girdle, not circular, instead of traditional circular pressotherapy girdles, or even its use as a splint, not circular, for selective compression after a sprain or closed joint trauma, instead of traditional circular splints or compression bandages.

In addition, in the case of its application on open wounds, the IVD has been conceived to extend its application at a distance from the skin edges of said wounds so that it even makes a bridge-tent effect on said edges, even in cases of complex wounds, deep and tunneled, that is, without being introduced or interposed said IVD between said edges; this, together with the already highlighted fact that the internal face of the internal component of the IVD is adherent to the skin, will maximize the approximation of said edges and, since it was neither introduced nor interposed between said edges of said wounds, it is going to make it possible that in some cases the closure of these wounds can even be achieved with a single application.

On the contrary, such cases of complex, deep and tunneled wounds, when treated with CVDs, the “in situ” cut of foam fragments of said CVDs is carried out to introduce them in said tunnels, adapting them as a filling material; and, in turn, said foam is trimmed and introduced interposed between said edges. Said trimming and filling presents multiple disadvantages:
Excessive consumption and waste of time,
Delay in the closing of said tunnels and wounds, since their filling with foam itself interferes/prevents their coaptation; therefore, frequent changes of said CVDs must be made, with progressively smaller filling fragments, to facilitate closure; which in turn consumes excessive time.
Risks of infection, due to the possibility of being inadvertently retained some of said fragments of said CVDs within said tunnels,
Bleeding risks, due to the growth of granulation tissue inside the pores of said fragments, especially if several days pass without changing said filling fragments, by tearing the neoformed granulation tissue when removing said filling fragments.
Loss of opportunity; since, as already mentioned, vacuum therapy with CVDs is limited to the lesional area and does not allow the treatment of the edema that exists also distant from said lesional area.

On the other hand, with the IVD, as has already been mentioned, the treatment of the tunnels of the complex lesions is mainly carried out through the expression and collapsing of said tunnels through the tangential and sagittal compression that the IVD exerts on the overlying surface of said tunnels. In addition, this can also be synergistically enhanced, promoting tissue adhesion and collapsing of said tunnels from their interior, by means of the tubular drain device shown in FIG. 2 of PCT/ES 2010/000221 of said IVD.

However, under the action of vacuum, the potential for tangential compression/retraction/entrainment of said IVD on the body surface, and, therefore, the drag/stretch potential of the skin underlying it, is limited by the potential of transversal/horizontal collapse of the pores of the external component of the IVD. Said potential can be increased by subjecting the IVD to tension/transverse stretching immediately before being applied on the body surface; with the added advantage that, as the internal component is adhesive, said tensile tension, when applied to the cutaneous surface, will exert a counter-traction that will contribute to approximate the edges of the wound even before applying the vacuum.

However, said stretching potential and subsequent transverse elongation of the pores of the external component is limited by the memory of the external component itself. There is the possibility of breaking this limit by increasing the space between the pores of the external component in multiple areas of said external component, if said external component was not monobloc, but was constituted by a plurality of adjacent foam fragments; so that, when subjected to transverse tension/stretch immediately before being applied to the body surface, said foam fragments would be separated, increasing transversely the spaces between them. That is, the separation of the walls of said pores at the level of said spaces between fragments would occur, which would produce an effect similar to that of an enlargement of the pores of the external component, since said spaces between fragments would behave in practice as giant macropores.

In addition, there would be the possibility of further increasing said retraction potential of the new vacuum dressing, after application of the vacuum, if said foam fragments were separated even before applying tensile stress on them before being placed on the skin. It would therefore be desirable, and this is the essential objective of the present invention, the creation, of a new vacuum dressing in which the internal component of the new vacuum dressing has, distributed over its entire surface, multiple areas devoid of external component. With the advantages of a greater retraction potential of the dressing and a greater homogeneity in the distribution of the vacuum through said External Component; which would lead to an enhancement of compression and drainage.

Also, said retraction potential could increase more if said areas devoid of external component were interconnected with each other.

Also, if the external surface of said foam fragments of the external component, that is, the surface of the fragments of the external component that is in contact with the sealing sheet, on which the suction pipe connected to the pump vacuum is coupled, was not flat, that is to say convex, would allow, together with a greater approximation/retraction potential, that is to say of tangential compression, a greater drainage potential due to the tent effect of said sealing sheet on the non-planar external surface of said foam fragments.

And, also, in the case of the application of said new dressing on a very large body area, by favoring a greater homogeneity in the distribution of the vacuum through said external component, it would present, among other additional advantages, that of not needing to couple more than one suction tube on said sealing sheet, to achieve said homogeneity in the distribution of the vacuum.

However, on the part of the applicant, the existence of a vacuum dressing that exhibits such characteristics is unknown,

Thus, the new vacuum dressing that the present invention proposes, is configured as a novelty within its field of application, since, according to its implementation, the objectives previously indicated as suitable are satisfactorily achieved.

### DESCRIPTION OF THE INVENTION

The vacuum dressing for the enhancement of compression and drainage in vacuum therapy, which is the object of the present invention, is one of those, which are sealed by an adhesive sheet on which is coupled a suction tube connected to a vacuum pump, and comprises under the aforementioned adhesive sheet an external component, which is a water-permeable open pore foam, and an internal component, which is attached to said external component, and which comprises at least one outer layer, which is attached to said external component, permeable to water vapor and impermeable to water and an internal layer adherent to the skin and intended to contact the body surface. The internal component may comprise a hydrophilic intermediate layer. Said outer component is constituted by a plurality of foam fragments adjacent to each other and distributed over the surface of the internal component. In the spaces between said foam fragments, a plurality of areas devoid of external component are generated in said internal component, and where the shape of the external surface, intended to be in contact with said adhesive sheet, of each of the fragments of foam is convex.

### BRIEF DESCRIPTION OF FIGURES

To complement the description that is being made and in order to help a better understanding of the characteristics of the invention, the present descriptive memory is accompanied, as an integral part thereof, by a set of plans, in which illustrative and not limitative the following has been represented:

FIG. 1: shows a view of an elevation of the vacuum dressing being stretched.

FIG. 2: shows the vacuum dressing, which, after being stretched, is going to be applied with a bridge-tent effect on a dehiscent wound, both on the edges of the wound and distant from it.

FIG. 3 shows that after applying the vacuum dressing on a dehiscent wound and since the internal surface of the internal component is adherent, it has allowed an approximation of the edges of the wound even before applying the vacuum,

FIG. 4: shows a view of the plant of the vacuum dressing in which the areas devoid of external component are isolated from one another.

FIG. 5: shows a view of the plant of the vacuum dressing in which the areas devoid of external component are connected to each other.

FIG. 6: shows a representation of the vacuum dressing object of the invention, applied on a depressed body surface, with loss of substance, as well as on the perilesional surface and also on the distant skin.

FIG. 7: shows a representation of the vacuum dressing object of the invention, also applied both on the bed of a wound (in this case an open abdomen) and distant from it, on the side walls of the trunk.

### PREFERRED EMBODIMENTS

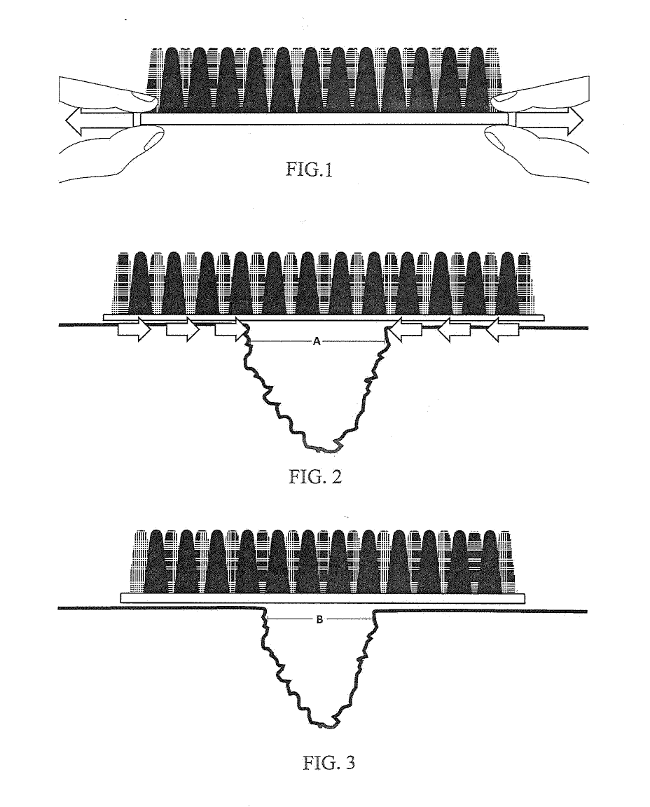

FIGS. 1 to 3 show a schematic representation of the application process of the vacuum dressing to a dehiscent wound, where it is first pulled, and therefore the dressing is stretched, and when applied to the wound, as the the outer layer of the internal component is adherent to the skin, before applying the vacuum, approximates the edges of the wound reducing the distance A to a smaller distance B.

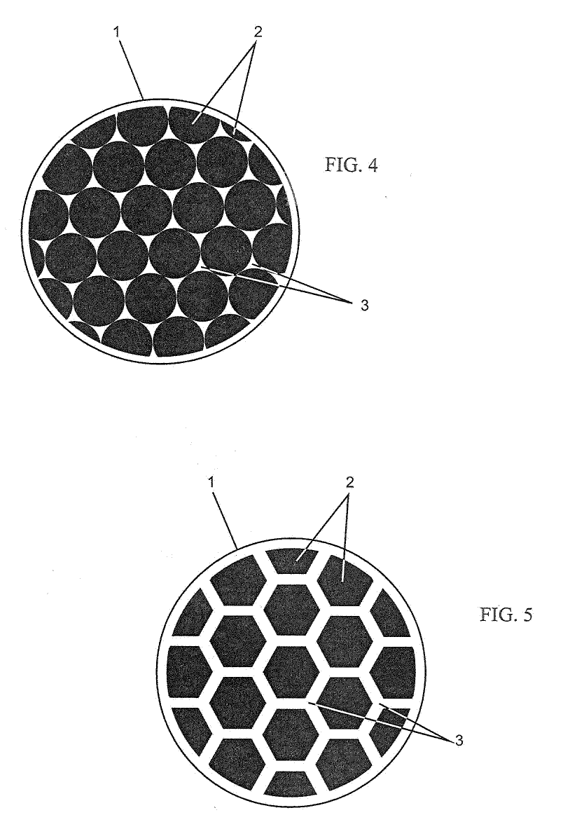

FIGS. 4 and 5 show two preferred embodiments of the vacuum dressing (1) which is the object of the present invention. In FIG. 4, the foam fragments (2) of the vacuum dressing have a conical shape and their bases are in contact with each other, whereby the areas (3) devoid of external component are isolated from each other, whereas in FIG. 5 the foam fragments (2) have a pyramidal shape, whose base can be hexagonal, and said bases are not in contact with each other so that the areas (3) devoid of external component are interconnected with each other.

The shape of at least one of the fragments can be an elliptic, hemispherical, conical or pyramidal paraboloid.

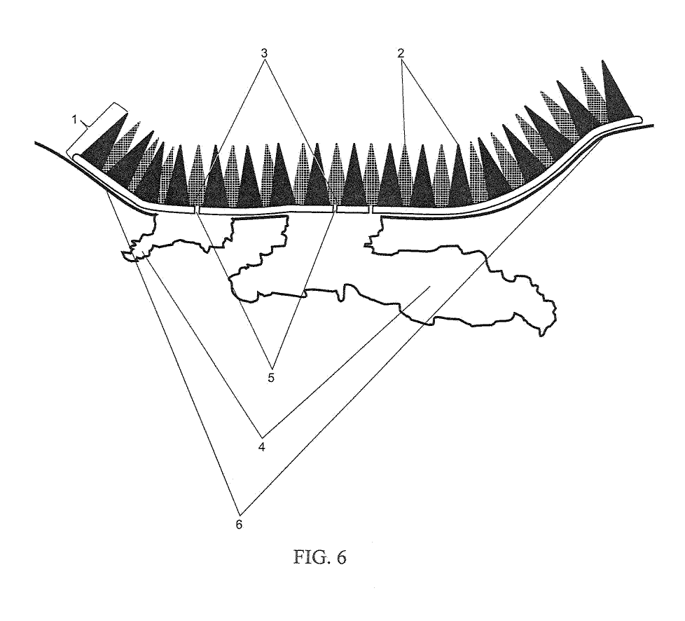

FIG. 6 shows a representation of the new dressing (1) object of the invention; said external component of said vacuum dressing is not monobloc, but is constituted by multiple adjacent foam fragments (2); the outer surface of said foam fragments of said external component being conical or pyramidal. Said new vacuum dressing is applied on a depressed body surface, in which two wounds with loss of substance, adjacent and with tunneling, coincide (4). Said vacuum dressing has one or more through holes (5) in some of the areas (3) devoid of external component, which may have been made “in situ” at the time before its application exclusively where said areas are coincident/overlapping with the beds of said wounds or coming from the factory. Said vacuum dressing, with said internal component, is also applied at a distance (6) from said wounds, to allow/promote remote compression, both sagittally and tangentially; what will favor, among others, the collapse/resolution of said tunneling and the reduction of the diameter of said wounds. Said vacuum dressing, has not required to be trimmed or interposed between the edges of said wound; so it will not limit the approximation of edges of said wound, but will enhance the remote compression favoring drainage and approximation of said edges. And, in addition, although it is also a “giant” dressing it will not be necessary to fit more than one suction tube on said sealing sheet. All this will lead to a saving of time, a gain in effectiveness opportunity and greater comfort for the patient.

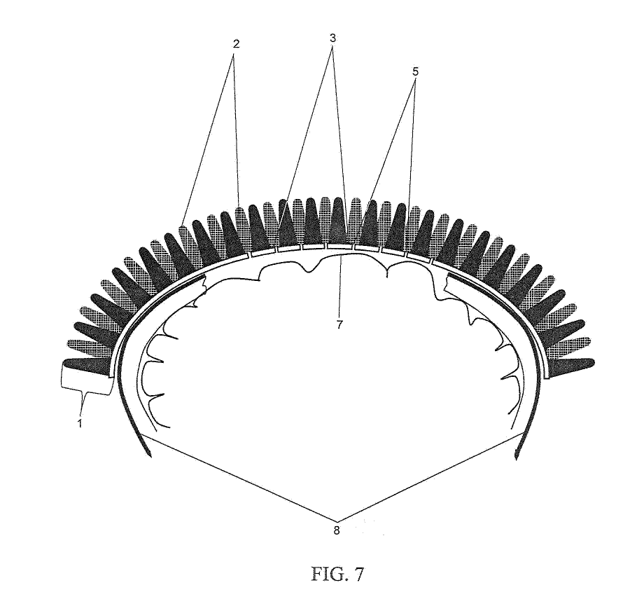

FIG. 7 shows a representation of the new dressing (1) object of the invention; said external component of said vacuum dressing is not monobloc, but is constituted by multiple adjacent foam fragments; the outer surface of said fragments of said external component being hemispherical or paraboloid. Said new dressing is applied both on the bed of an open abdomen (7), and at a distance from said bed, on the lateral walls of the trunk (8). Said vacuum dressing has one or more through holes (5) at the level of the central areas (3) devoid of external component of said internal component, either performed “in situ” at the time before its application, or in a manufactured manner. Said bare perforated areas are those that are to be superposed on the intestinal loops, to enhance said drainage and, at the same time, to protect/isolate said loops, even more efficiently, from contact with said external component; said perforations being exclusively at the level of said bare areas, devoid of said external component. It is also important to point out that said vacuum dressing, unlike the conventional vacuum dressings used in the therapy of the open abdomen, has also not needed to be trimmed or interposed between the edges of said wound; so it will not limit the approximation of edges of said wound and, also, despite being a “giant” dressing, it will not need to fit more than one aspiration tube on said sealing sheet. All this will also lead to said time saving, said gain in effectiveness/opportunity and said greater comfort for the patient.

Having sufficiently described the nature of the present invention, as well as the way to put it into practice, it is not considered necessary to extend its explanation so that any expert in the field understands its scope and the advantages derived from it, stating that, within its essentiality, it may be carried out in other forms of realization that differ in detail from that indicated by way of example, and which will also achieve the protection that is sought provided that it is not altered, changed or modified its fundamental principle.

1. Vacuum dressing for the enhancement of compression and drainage in vacuum therapy, of those, which are sealed by an adhesive sheet on which is coupled a suction tube connected to a vacuum pump, and which comprise below of said adhesive sheet an external component, which is a water-permeable open pore foam, and an internal component, which is attached to said external component, and which comprises at least one outer layer, which is attached to said external component, water vapor permeable and impermeable to water and an internal layer adherent to the skin and intended to contact the body surface, characterized in that said external component is constituted by a plurality of foam fragments adjacent to each other and distributed over the surface of the internal component and in the spaces between said foam fragments a plurality of areas devoid of external component is generated in said internal component.

2. Vacuum dressing for the enhancement of compression and drainage in vacuum therapy, of those, which are sealed by an adhesive sheet on which is coupled a suction tube connected to a vacuum pump, and which comprise below of said adhesive sheet an external component, which is a water-permeable open pore foam, and an internal component, which is attached to said external component, and which comprises at least one outer layer, which is attached to said external component, water vapor permeable and impermeable to water and an internal layer adherent to the skin and intended to contact the body surface, characterized in that said external component is constituted by a plurality of foam fragments adjacent to each other and distributed over the surface of the internal component, in the spaces between said foam fragments a plurality of areas devoid of external component is generated in said internal component, and where the shape of the external surface of each of the foam fragments, intended to be in contact with said adhesive sheet, is convex.

3. Vacuum dressing for the enhancement of compression and drainage, according to claim 1, characterized in that the fragments are arranged in such a way on the internal component that the areas devoid of external component are interconnected with each other.

4. Vacuum dressing for the enhancement of compression and drainage, according to claim 3, characterized in that a hydrophilic intermediate layer is arranged between the outer layer and the internal layer of the internal component.

5. Vacuum dressing for the enhancement of compression and drainage, according to claim 1, characterized in that the fragments are arranged in such a way on the internal component that the areas devoid of external component are isolated from each other.

6. Vacuum dressing for the enhancement of compression and drainage, according to claim 5, characterized in that a hydrophilic intermediate layer is arranged between the outer layer and the internal layer of the internal component.

7. Vacuum dressing for the enhancement of compression and drainage, according to claim 2, characterized in that the fragments are arranged in such a way on the internal component that the areas devoid of external component are interconnected with each other.

8. Vacuum dressing for the enhancement of compression and drainage, according to claim 7, characterized in that at least one of the fragments has an elliptic paraboloid shape.

9. Vacuum dressing for the enhancement of compression and drainage, according to claim 7, characterized in that at least one of the fragments has a conical shape.

10. Vacuum dressing for the enhancement of compression and drainage, according to claim 7, characterized in that at least one of the fragments has a hemispherical shape.

11. Vacuum dressing for the enhancement of compression and drainage, according to claim 7, characterized in that at least one of the fragments has a pyramidal shape.

12. Vacuum dressing for the enhancement of compression and drainage, according to claim 7, characterized in that a hydrophilic intermediate layer is arranged between the outer layer and the internal layer of the internal component.

13. Vacuum dressing for the enhancement of compression and drainage, according to claim 7, characterized in that at least one of the areas devoid of external component has one or more through holes.

14. Vacuum dressing for the enhancement of compression and drainage, according to claim 2, characterized in that the fragments are arranged in such a way on the internal component that the areas devoid of external component are isolated from each other.

15. Vacuum dressing for the enhancement of compression and drainage, according to claim 14, characterized in that at least one of the fragments has an elliptic paraboloid shape.

16. Vacuum dressing for the enhancement of compression and drainage, according to claim 14, characterized in that at least one of the fragments has a conical shape.

17. Vacuum dressing for the enhancement of compression and drainage, according to claim 14, characterized in that at least one of the fragments has a hemispherical shape.

18. Vacuum dressing for the enhancement of compression and drainage, according to claim 14, characterized in that at least one of the fragments has a pyramidal shape.

19. Vacuum dressing for the enhancement of compression and drainage, according to claim 14, characterized in that a hydrophilic intermediate layer is arranged between the outer layer and the internal layer of the internal component.

20. Vacuum dressing for the enhancement of compression and drainage, according to claim 14, characterized in that at least one of the areas devoid of external component has one or more through holes.
